(19) United States
(12) Patent Application Publication
(10) Pub. No.: US 2012/0108779 A1
(43) Pub. Date: May 3, 2012

(54) METHOD FOR PRODUCING POLYOLS ON THE BASIS OF RENEWABLE RESOURCES

(75) Inventors: Andreas Kunst, Ludwigshafen (DE); Michael Schelper, Ludwigshafen (DE); Joaquim Henrique Teles, Otterstadt (DE); Berend Eling, Lemfoerde (DE); Jenny Reuber, Mannheim (DE); Gerd-Dieter Tebben, Mannheim (DE)

(73) Assignee: BASF SE, Ludwigshafen (DE)

(21) Appl. No.: 13/381,116

(22) PCT Filed: Jul. 9, 2010
(86) PCT No.: PCT/EP2010/059850

§ 371 Date: Dec. 28, 2011

(30) Foreign Application Priority Data
Jul. 10, 2009 (EP) 09165143.0

(51) Int. Cl. C08G 18/28; C07C 51/27

(52) U.S. Cl. 528/85; 554/132

(57) ABSTRACT

The invention provides a method for producing polyols, comprising the steps of
a) reacting unsaturated natural fats, unsaturated natural fatty acids and/or fatty acid esters with dinitrogen monoxide,
b) reacting the product obtained in step a) with hydrogen using a heterogeneous catalyst.

The invention provides a method for producing polyols on the basis of natural oils, more particularly for the preparation of polyurethanes.

Polyurethanes are used in numerous technical fields. They are customarily prepared by reacting polyisocyanates with compounds having at least two hydrogen atoms that are reactive with isocyanate groups, in the presence of blowing agents and, optionally, of catalysts and customary auxiliaries and/or adjuvants.

More recent times have seen an increase in the significance of polyurethane starting components based on renewable raw materials. More particularly in the case of the compounds having at least two hydrogen atoms that are reactive with isocyanate groups, it is possible for natural oils and fats to be employed, which are customarily modified chemically before being used in polyurethane applications, in order to introduce at least two hydrogen atoms that are reactive with isocyanate groups. Generally speaking, during the chemical modifications, natural fats and/or oils are hydroxyl-functionalized, and optionally modified in one or more further steps. Examples of applications of hydroxyl-functionalized fat derivatives and/or oil derivatives in PU systems include WO 2006/116456 and WO2007/130524, for example.

The reactive hydrogen atoms that are needed for use in the polyurethane industry have to be introduced as described above by means of chemical methods into most of the naturally occurring oils. For this purpose, in accordance with the state of the art, methods exist, substantially, that utilize the double bonds that occur in the fatty acid esters of many oils. Firstly, fats can be oxidized to the corresponding fatty epoxides or fatty acid epoxides by reaction with percarboxylic acids in the presence of a catalyst. The subsequent acid-catalyzed or base-catalyzed ring opening of the oxirane rings in the presence of alcohols, water, caroboxylic acids, halogens or hydrogen halides leads to formation of hydroxyl-functionalized fats or fatty derivatives, respectively, described in WO 2007/127379 and US 2008076901, for example. The disadvantage of this method is that the first reaction step (expoxidation) requires use of highly corrosion-resistant materials, this reaction step being carried out industrially using corrosive performic acid or using peracetic acid. After production, furthermore, the dilute percarboxylic acid obtained must, for an economic process, be concentrated again by distillation and recycled, and this necessitates the use of corrosion-resistant distillation apparatus, which is therefore more energy-intensive and costly.

Another possibility for hydroxy-functionalization is to subject the unsaturated fat or fatty acid derivative in the first reaction step, in the presence of a catalyst comprising cobalt or comprising rhodium, first to hydroformylation with a mixture of carbon monoxide and hydrogen (synthesis gas), and subsequently to hydrogenation of the aldehyde functions introduced with this reaction step to hydroxyl groups (cf. WO 2006/12344 A1 or else J. Mol. Cat. A, 2002, 184, 65 and J. Polym. Environm. 2002, 10, 49), using an appropriate catalyst (e.g., Raney nickel). With this reaction pathway, however, it must be borne in mind that the first reaction step, the hydroformylation, as well requires at least the use of a catalyst and a solvent, which for an economic preparation must likewise be recovered again and processed or regenerated.

EP1170274A1 describes a method for producing hydroxyl oils by oxidizing unsaturated oils in the presence of atmospheric oxygen. Disadvantages are that with this method the degrees of functionalization obtained are not high, and that the reactions have to take place at high temperatures, leading to partial decomposition of the fat structure. A further possibility of introducing hydroxyl functions into fats is to cleave fat or the fatty derivative in the presence of ozone, and then to carry out reduction to form the hydroxyl-fat derivative (cf. Biomacromolecules 2005, 6, 713; J. Am. Oil Chem. Soc. 2005, 82, 653 and J. Am. Oil Chem. Soc. 2007, 84, 173). This procedure as well has to take place in a solvent, and is carried out customarily at low temperatures (−10 to 0° C.), likewise resulting in comparatively high manufacturing costs. The safety characteristics of this procedure, moreover, require the costly provision of safety measures, such as measurement and control technology or compartmentalization.

In Adv. Synth. Catal. 2007, 349, 1604, the ketonization of fats by means of laughing gas is described. The ketone groups can be converted into hydroxyl groups using homogeneous catalysts. However, there is no reference at all to the further-processing of these products.

One possibility for preparing polyols on the basis of renewable raw materials for polyurethanes is to react unsaturated, naturally occurring fats such as soybean oil, sunflower oil, rapeseed oil, etc., for example, or corresponding fatty derivatives such as fatty acids or their monoesters, by corresponding derivatization, to give hydroxy-functionalized fats and fatty acid derivatives, respectively. These materials can be used for the corresponding PU application either directly or, alternatively, after extra addition of alkaline oxides onto the OH functions in the hydroxy-functionalized fat or fatty derivative. Examples of the reaction of hydroxy-fatty derivatives with alkylene oxides and the use of the reaction products in polyurethane applications can be found in WO 2007/143135 and EP1537159, for example. The addition reaction here takes place in the majority of cases by means of catalysts known as double metal cyanide catalysts.

It was an object of the present invention to provide polyols based on renewable raw materials, more particularly based on natural fats and fatty acid derivatives, for polyurethane applications, which are available inexpensively and in the case of which a very simple adaptation to the reaction parameters makes it possible to cover a very wide variety of functionalities, making the products, therefore, available for a broad sphere of application. More particularly it ought to be possible to produce the oils and fats by a simple method without the use of expensive raw materials (catalysts and solvents). At the same time, it ought to be possible to remove catalysts from the reaction product in a simple way.

This object has been achieved by subjecting unsaturated natural fats such as soybean oil, sunflower oil, rapeseed oil, castor oil or corresponding fatty acid derivatives to oxidation to form ketonized fats and fatty acid derivatives in a first step in the presence of dinitrogen monoxide, also referred to as laughing gas, and, in a further reaction step, subjecting these products to reduction in the presence of hydrogen and a heterogeneous catalyst, to give hydroxyl-fats.

The invention provides, accordingly, a method for producing polyols based on renewable raw materials, comprising the steps of

a) reacting unsaturated natural fats, unsaturated natural fatty acids and/or fatty acid esters with dinitrogen monoxide,

b) reacting the product obtained in step a) with hydrogen using a heterogeneous catalyst.

These materials can be employed directly as a polyol component across a very wide variety of applications, as for example in the corresponding PU application.

The natural, unsaturated fats are preferably selected from the group containing castor oil, grapeseed oil, black cumin oil, pumpkin seed oil, borage seed oil, soybean oil, wheatgerm oil, rapeseed oil, sunflower oil, peanut oil, apricot kernel oil, pistachio oil, almond oil, olive oil, macadamia nut oil, avocado oil, seabuckthorn oil, sesame oil, hemp oil, hazelnut oil, primula oil, wild rose oil, safflower oil, walnut oil, palm oil, fish oil, coconut oil, tall oil, corngerm oil, linseed oil.

Preferred fatty acids and fatty acid esters are those selected from the group containing myristoleic acid, palmitoleic acid, oleic acid, vaccenic acid, petroselinic acid, gadoleic acid, erucic acid, nervonic acid, linoleic acid, α- and γ-linolenic acid, stearidonic acid, arachidonic acid, timnodonic acid, clupanodonic acid, and cervonic acid, and also esters thereof.

As fatty acid esters it is possible to use not only fully esterified but also partly esterified monohydric or polyhydric alcohols. Monohydric or polyhydric alcohols contemplated include methanol, ethanol, propanol, isopropanol, butanol, ethylene glycol, propylene glycol, diethylene glycol, dipropylene glycol, glycerol, trimethylolpropane, pentaerythritol, sorbitol, sucrose and mannose.

Particular preference is given to the natural, unsaturated fats selected from the group containing castor oil, soybean, palm, sunflower, and rapeseed oil. Use is made more particularly of soybean, palm, sunflower, and rapeseed oil. These compounds are used on the industrial scale not least for the production of biodiesel as well.

Besides the oils stated it is also possible to use those oils obtained from genetically modified plants, having a different fatty acid composition. Besides the stated oils it is likewise possible, as described above, to use the corresponding fatty acids or fatty acid esters.

The reaction steps a) and b) can be carried out independently of one another and optionally also separately in terms of time and place. It is possible, however, to carry out three method steps immediately following one another. In this context it is also possible to carry out the method entirely continuously.

Step a) is carried out preferably under superatmospheric pressure, more particularly in a pressure range from 10 to 300 bar, and at elevated temperature, more particularly in a temperature range from 200 to 350° C. Here it is possible to use the oil or fat in bulk or in solutions with suitable solvents, such as cyclohexane, acetone or methanol. The reaction can take place in a stirred reactor of any desired design or in a tube reactor; reaction in any desired other reactor systems is possible in principle. The laughing gas used can be used as the pure substance or as a mixture with gases that are inert under the reaction conditions, such as nitrogen, helium, argon or carbon dioxide. The amount of the inert gases in this case is not more than 50% by volume.

For further processing of the reaction mixture after the end of the reaction, the reaction mixture is cooled, the solvent is removed if necessary, by means of distillation or extraction, for example, and the product is supplied to step b), with or without further work-up.

The reaction product from step a) is hydrogenated in step b). This too takes place in accordance with customary and known methods. For this purpose, the preferably purified organic phase from step a) is reacted with hydrogen, preferably in the presence of a suitable solvent. For this purpose the organic phase, under a pressure of 50 to 300 bar, more particularly at 90 to 150 bar, and at a temperature of 50 to 250° C., more particularly 50 to 120° C., is reacted in the presence of hydrogenation catalysts. Hydrogenation catalysts are heterogeneous catalysts. Preference is given to using catalysts comprising ruthenium. Apart from ruthenium, the catalysts may also comprise other metals, examples being metals from groups 6-11 such as nickel, cobalt, copper, molybdenum, palladium or platinum, for example.

The catalysts are preferably applied on supports. Supports which can be used are the customary supports, such as aluminum oxide or zeolites. In one preferred embodiment of the invention, carbon is used as support material.

The catalysts may be water-moist. The hydrogenation is carried out preferably in a fixed bed.

Following the hydrogenation, the organic solvents, the catalyst and, if necessary, water are removed. The product is purified where necessary.

Depending on the nature of the fat or fatty derivative used in procedural step a), the polyols from procedural step b) have an average functionality of 2 to 6, more particularly of 2 to 4, and a hydroxyl number in the range between 50 and 300 mg KOH/g. The structures are suitable more particularly for producing polyurethanes, more particularly for flexible polyurethane foams, rigid polyurethane foams, and polyurethane coatings. In the production of rigid polyurethane foams and polyurethane coatings it is in principle also possible to use those polyols which have not been addition-reacted with alkylene oxides—in other words, polyols based on renewable raw materials and prepared by implementation only of method steps a) and b). In the course of the production of flexible polyurethane foams, compounds of this kind, on account of their low chain lengths, result in an unwanted crosslinking, and are therefore less suitable.

The polyurethanes are produced by reacting the polyether alcohols, prepared by the method of the invention, with polyisocyanates.

The polyurethanes of the invention are prepared by reaction of polyisocyanates with compounds having at least two hydrogen atoms that are reactive with isocyanate groups. In the case of the production of the foams, the reaction takes place in the presence of blowing agents.

The starting compounds used are subject to the following specific remarks:

Polyisocyanates contemplated include the conventional aliphatic, cycloaliphatic, araliphatic, and, preferably, aromatic polyfunctional isocyanates.

Specific examples include the following: alkylene diisocyanates having 4 to 12 carbon atoms in the alkylene radical, such as, for example, hexamethylene 1,6-diisocyanate; cycloaliphatic diisocyanates, such as, for example, cyclohexane-1,3- and -1,4-diisocyanate, and also any desired mixtures of these isomers, hexahydrotolylene 2,4- and 2,6-diisocyanate, and also the corresponding isomer mixtures, dicyclohexylmethane 4,4′-, 2,2′- and 2,4′-diisocyanate, and also the corresponding isomer mixtures, araliphatic diisocyanates, such as, for example, xylylene 1,4-diisocyanate and xylylene diisocyanate isomer mixtures, but preferably aromatic diisocyanates and polyisocyanates, such as, for example, tolylene 2,4- and 2,6-diisocyanate (TDI) and the corresponding isomer mixtures, diphenylmethane 4,4′-, 2,4′- and 2,2′-diisocyanate (MDI) and the corresponding isomer mixtures, mixtures of diphenylmethane 4,4′- and 2,4′-diisocyanates, polyphenyl-polymethylene polyisocyanates, mixtures of diphenylmethane 4,4′-, 2,4′- and 2,2′-diisocyanates and polyphenyl-polymethylene polyisocyanates (crude MDI) and mixtures of crude MDI and tolylene diisocyanates. The organic diisocyanates and polyisocyanates can be used individually or in the form of mixtures.

Use is frequently also made of what are called modified polyfunctional isocyanates, these being products obtained by chemical reaction of organic diisocyantes and/or polyisocyanates. Examples include diisocyanates and/or polyisocyanates containing isocyanurate groups and/or urethane groups. Specific examples contemplated include organic, preferably aromatic, polyisocyanates containing urethane groups, having NCO contents of 33% to 15% by weight, preferably of 31% to 21% by weight, based on the total weight of the polyisocyanate.

The polyols produced by the method of the invention can be used in combination with other compounds having at least two hydrogen atoms that are reactive with isocyanate groups.

As compounds having at least two isocyanate-reactive hydrogen atoms, which can be used together with the polyols produced by the method of the invention, polyether alcohols and/or polyester alcohols are employed more particularly.

In the case of the production of rigid polyurethane foams, it is usual to use at least one polyether alcohol which has a functionality of at least 4 and a hydroxyl number of greater than 250 mg KOH/g.

The polyester alcohols used together with the polyols produced by the method of the invention are prepared generally by condensation of polyfunctional alcohols, preferably diols, having 2 to 12 carbon atoms, preferably 2 to 6 carbon atoms, with polyfunctional carboxylic acids having 2 to 12 carbon atoms, examples being succinic acid, glutaric acid, adipic acid, suberic acid, azelaic acid, sebacic acid, decanedicarboxylic acid, maleic acid, fumaric acid, and, preferably, phthalic acid, isophthalic acid, terephthalic acid, and the isomeric naphthalenedicarboxylic acids.

The polyether alcohols used together with the polyols produced by the method of the invention generally have a functionality of between 2 and 8, more particularly 4 to 8.

Use is made more particularly as polyhydroxyl compounds of polyether polyols, which are prepared by known methods, as for example by anionic polymerization of alkylene oxides in the presence of alkali metal hydroxides.

Alkylene oxides used are preferably ethylene oxide and 1,2-propylene oxide. The alkylene oxides can be used individually, or alternately in succession or as mixtures.

Examples of starter molecules, contemplated include the following: water, organic dicarboxylic acids, such as succinic acid, adipic acid, phthalic acid, and terephthalic acid, for example, aliphatic and aromatic, optionally N-mono-, N,N- and N,N′-dialkyl substituted diamines having 1 to 4 carbon atoms in the alkyl radical, such as optionally mono- and dialkyl substituted ethylenediamine, diethylene triamine, triethylene tetramine, 1,3-propylene diamine, 1,3- and/or 1,4-butylenediamine, 1,2-, 1,3-, 1,4-, 1,5- and 1,6-hexamethylenediamine, aniline, phenylenediamines, 2,3-, 2,4-, 3,4- and 2,6-tolylenediamine, and 4,4′-, 2,4′- and 2,2′-diaminodiphenylmethane, for example.

Further starter molecules contemplated include the following: alkanolamines, such as ethanolamine, N-methyl- and N-ethylethanolamine, for example, dialkanolamines, such as diethanolamine, N-methyl- and N-ethyldiethanolamine, for example, and trialkanolamines such as triethanolamine, for example, and ammonia.

Use is made additionally of polyhydric alcohols, more particularly dihydric and/or trihydric alcohols, such as ethanediol, propane-1,2- and -1,3-diol, diethylene glycol, dipropylene glycol, butane-1,4-diol, hexane-1,6-diol, glycerol, pentaerythritol, sorbitol, and sucrose, polyhydric phenols, such as 4,4′-dihydroxydiphenylmethane and 2,2-bis(4-hydroxyphenyl)propane, for example, resoles, such as, for example, oligomeric condensation products of phenol and formaldehyde, and Mannich condensates of phenols, formaldehyde and dialkanolamines, and also melamine.

The polyetherpolyols possess a functionality of preferably 3 to 8 and more particularly 3 and 6, and hydroxyl numbers of preferably 120 mg KOH/g to 770 mg KOH/g and more particularly 240 mg KOH/g to 570 mg KOH/g.

The compounds having at least two hydrogen atoms that are reactive with isocyanate groups also include the optionally co-used chain extenders and crosslinkers. For modifying the mechanical properties, however, the addition of difunctional chain extenders, crosslinking agents with a functionality of three or more, or, optionally, mixtures thereof, may prove advantageous. Chain extenders and/or crosslinking agents used are preferably alkanolamines and more particularly diols and/or triols having molecular weights of less than 400, preferably 60 to 300.

Where chain extenders, crosslinking agents or mixtures thereof are employed in producing the polyurethanes, they are employed usefully in an amount from 0% to 20% by weight, preferably 2% to 5% by weight, based on the weight of the compounds having at least two hydrogen atoms that are reactive with isocyanate groups.

As blowing agent it is possible, for example, to use water, which on reaction with isocyanate groups eliminates carbon dioxide. Instead of, but preferably in combination with, water it is also possible to use what are called physical blowing agents. These are compounds which are inert toward the ingredient components and are usually liquid at room temperature, but which vaporize under the conditions of the urethane reaction. The boiling point of these compounds is preferably below 110° C., more particularly below 80° C. The physical blowing agents also include inert gases, which are introduced into the ingredient components or dissolve therein, examples being carbon dioxide, nitrogen or noble gases.

The compounds liquid at room temperature are generally selected from the group containing alkanes and/or cycloalkanes having at least 4 carbon atoms, dialkyl ethers, esters, ketones, acetyls, fluoroalkanes having 1 to 8 carbon atoms, and tetraalkylsilanes having 1 to 3 carbon atoms in the alkyl chain, more particularly tetramethylsilane.

Examples include propane, n-butane, isobutane, and cyclobutane, n-pentane, isopentane, and cyclopentane, cyclohexane, dimethyl ether, methyl ethyl ether, methyl butyl ether, methyl formate, acetone and also fluoroalkanes which can be broken down in the troposphere and are therefore not harmful to the ozone layer, such as trifluoromethane, difluoromethane, 1,1,1,3,3-pentafluorobutane, 1,1,1,3,3-pentafluoropropane, 1,1,1,2-tetrafluoroethane, difluoroethane, and heptafluoropropane. The stated physical blowing agents may be used alone or in any desired combinations with one another.

Catalysts used are more particularly compounds which sharply accelerate the reaction of the isocyanate groups with the groups that are reactive with isocyanate groups. Use is made more particularly of organometallic compounds, preferably organotin compounds, such as tin(II) salts of organic acids.

As catalysts it is additionally possible to use strongly basic amines. Examples thereof are secondary aliphatic amines, imidazoles, amidines, triazines, and alkanolamines. Depending on requirement, the catalysts can be used alone or in any desired mixtures with one another.

Auxiliaries and/or adjuvants employed are the substances that are known per se for this purpose, examples being surface-active substances, foam stabilizers, cell regulators, fillers, pigments, dyes, flame retardants, hydrolysis inhibitors, antistats, and agents with fungistatic and bacteriostatic activity.

More detailed information on the starter materials, blowing agents, catalysts, and auxiliaries and/or adjuvants used for implementing the method of the invention are found in, for example, Kunststoffhandbuch, volume 7, “Polyurethane” Carl-Hanser-Verlag Munich, 1^{st} edition, 1966, 2^{nd} edition, 1983, and 3^{rd} edition, 1993.

The advantage of the method of the invention over epoxidization/ring opening and hydroformylation/hydrogenation is that the ketonization procedure does not require any solvents or any catalysts. Accordingly, comparatively inexpensive access to hydroxyl-functionalized fats and fatty acid derivatives is possible. In addition, the advantage exists that, through simple adaptation of the reaction conditions such as pressure, temperature, and residence time, functionalities can be adjusted in a very simple and targeted way, thereby providing access to materials which offer very broad possibilities for application, even going beyond polyurethane applications.

Relative to the epoxidization and the ozonolysis, this method offers the advantage of generating oligo-hydroxy fats which, while having a freely adjustable degree of hydroxylization, no longer contain any double bonds and therefore are no longer subject to the customary aging process of fats (oxidation of the DBs, “becoming rancid”). In the case of epoxidization and ozonolysis, this is accomplished only in the case of complete conversion—this, however, lays down the degree of functionalization.

In comparison to the hydroformylation, oxidation with laughing gas allows the production of material having complementary reactivity, since in this case it is exclusively secondary hydroxyl groups that are produced, whereas the hydroformylation produces primary OH groups.

The invention is illustrated using the examples below.

### EXAMPLE 1

### Oxidation of Soybean Oil with Laughing Gas

A steel autoclave with a capacity of 1.2 L was charged with 260 g of soybean oil, and then sealed and inertized with nitrogen. 50 bar of laughing gas were injected, the stirrer was set to 700 rpm and switched on, and the reaction mixture was subsequently heated to 220° C. After a running time of 22 hours, cooling took place to room temperature, the stirrer was switched off, and the autoclave was let down slowly to ambient pressure. Following removal of the solvent, the yellowish liquid discharge was analyzed.

Analytical data: bromine number 36 g bromine/100 g, carbonyl number 173 mg KOH/g, ester number 196 mg KOH/g, acid number 1.8 mg KOH/g. Elemental analysis: C=73.6%, H=10.8%, O=15.1%.

### EXAMPLE 2

### Oxidation of Soybean Oil with Laughing Gas

A steel autoclave with a capacity of 1.2 L was charged with 172 g of soybean oil and 172 g of cyclohexane, and then sealed and inertized with nitrogen. 20 bar of laughing gas were injected, the stirrer was set to 700 rpm and switched on, and the reaction mixture was subsequently heated to 220° C. After a running time of 36 hours, cooling took place to room temperature, the stirrer was switched off, and the autoclave was let down slowly to ambient pressure. Following removal of the solvent, the yellowish liquid discharge was analyzed.

Analytical data: bromine number 57 g bromine/100 g, carbonyl number 64 mg KOH/g, ester number 196 mg KOH/g, acid number 1.8 mg KOH/g. Elemental analysis: C=75.6%, H=11.5%, O=13.4%.

### EXAMPLE 3

### Oxidation of Soybean Oil with Laughing Gas in a Tube Reactor

In a tube reactor (internal volume 210 mL, residence time around 50 minutes) at 290° C. and 100 bar, 130 g/h of a mixture of 50% by weight soybean oil and 50% by weight cyclohexane were reacted with 45 g/h of laughing gas. The reaction discharge was let down into a vessel, the liquid fraction of the reaction discharge was cooled, and the cyclohexane was removed by distillation. The yellowish liquid discharge was analyzed. Analytical data: bromine number 54 g bromine/100 g, carbonyl number 81 mg KOH/g, ester number 199 mg KOH/g, acid number 2.6 mg KOH/g. Elemental analysis: C=75.0%, H=11.1%, O=13.7%.

The soybean oil used in all of the examples was a commercial product from Aldrich having a bromine number of 80 g bromine/100 g, a carbonyl number of 1 mg KOH/100 g, a saponification number of 192 mg KOH/g, and an acid number of <0.1 mg KOH/g. Elemental analysis showed C=77.6%, H=11.7%, O=11.0%.

### EXAMPLE 4

### Hydrogenation of the Oxidized Soybean Oil from Example 2

A 300 mL steel autoclave is charged with a solution of 20 g of oxidized soybean oil from Example 2 (carbonyl number 64 mg KOH/100 g, OH number<5 mg KOH/1 g, bromine number 57 g bromine/100 g) in 100 mL of tetrahydrofuran, together with 2 g of a water-moist, 5% ruthenium catalyst on a carbon support. Heating took place to 120° C., and 120 bar of hydrogen were injected. With these parameters, stirring was carried out for 12 hours. The reaction mixture was then cooled and let down. The discharge was filtered and the solvent is removed by distillation. Analysis of the solid (butterlike) residue gave an OH number of 64, a carbonyl number<5, and a bromine number of <5.

### EXAMPLE 5

### Hydrogenation of the Oxidized Soybean Oil from Example 3

A 300 mL steel autoclave was charged with a solution of 20 g of oxidized soybean oil (carbonyl number=81, bromine number=54) in 100 mL of tetrahydrofuran, together with 20 g of a water-moist, Al₂O₃-supported ruthenium catalyst (0.5%). Heating took place to 120° C., and 100 bar of hydrogen were injected. With these parameters, stirring was carried out for 12 hours. The reaction mixture was then cooled and let down. The reaction discharge was filtered and thereafter the solvent was removed by distillation. Analysis of the solid (butterlike) residue gave an OH number of 80, a carbonyl number<5, and a bromine number of <5.

The polyol from Example 5 was employed successfully in a polyurethane coating formula. In that case it was found that the coating is notable for a very high water repellency.

### EXAMPLE 6

### Hydrogenation of the Oxidized Soybean Oil from Example 1

A 300 mL steel autoclave was charged with a solution of 20 g of oxidized soybean oil from Example 1 (carbonyl number=173, OH number<5, bromine number=36) in 100 mL of tetrahydrofuran, together with 2 g of a water-moist, 5% ruthenium catalyst on a carbon support. Heating took place to 120° C., and 120 bar of hydrogen were injected. With these parameters, stirring was carried out for 12 hours. The reaction mixture was then cooled and let down. The discharge was filtered and thereafter the solvent was removed by distillation. Analysis of the solid (butterlike) residue gave an OH number of 170, a carbonyl number<5, and a bromine number of <5.

The polyol from Example 6 was employed in a rigid polyurethane foam formula. In that case it was found that the system was notable for outstanding compatibility with the pentane blowing agent used.

1. A method for producing a polyol, the method comprising
a) reacting a raw material with dinitrogen monoxide to form a product, and b) reacting the product with hydrogen in the presence of a heterogeneous catalyst to form the polyol,
wherein the raw material comprises at least one of an unsaturated natural fat, an unsaturated natural fatty acid, and an unsaturated natural fatty acid ester.

2. The method of claim 1, wherein the raw material is at least one selected from the group consisting of castor oil, grapeseed oil, black cumin oil, pumpkin seed oil, borage seed oil, soybean oil, wheatgerm oil, rapeseed oil, sunflower oil, peanut oil, apricot kernel oil, pistachio oil, almond oil, olive oil, macadamia nut oil, avocado oil, seabuckthorn oil, sesame oil, hemp oil, hazelnut oil, primula oil, wild rose oil, safflower oil, walnut oil, palm oil, fish oil, coconut oil, tall oil, corngerm oil, and linseed oil.

3. The method of claim 1, wherein the raw material is at least one selected from the group consisting of myristoleic acid, palmitoleic acid, oleic acid, vaccenic acid, petroselinic acid, gadoleic acid, erucic acid, nervonic acid, linoleic acid, α- and γ-linolenic acid, stearidonic acid, arachidonic acid, timnodonic acid, clupanodonic acid, and cervonic acid, and esters thereof.

4. The method of claim 1, wherein the raw material is at least one selected from the group consisting of soybean oil, palm oil, sunflower oil, rapeseed oil and castor oil.

5. The method of claim 1, wherein the dinitrogen monoxide comprises an inert gas.

6. The method of claim 1, wherein the heterogeneous catalyst comprises ruthenium.

7. The method of claim 1, wherein the heterogeneous catalyst is applied on a support.

8. The method of claim 7, wherein the support is carbon.

9. The method of claim 1, wherein the heterogeneous catalyst is in the form of a fixed bed.

10. A polyol obtained by the method of claim 1.

11. A polyurethane obtained from the polyol of claim 10.

12. A method for preparing a polyurethane the method comprising reacting at least one polyisocyanate with a compound having at least two hydrogen atoms that are reactive with isocyanate groups, wherein the compound comprises the polyol of claim 10.

13. A polyol obtained by the method of claim 3.

14. A polyurethane obtained from the polyol of claim 13.

15. A method for preparing a polyurethane, the method comprising reacting at least one polyisocyanate with a compound having at least two hydrogen atoms that are reactive with isocyanate groups, wherein the compound comprises the polyol of claim 13.

16. A polyol obtained by the method of claim 4.

17. A polyurethane obtained from the polyol of claim 13.

18. A method for preparing a polyurethane, the method comprising reacting at least one polyisocyanate with a compound having at least two hydrogen atoms that are reactive with isocyanate groups, wherein the compound comprises the polyol of claim 13.
